(19) United States
(12) Patent Application Publication
(10) Pub. No.: US 2010/0145246 A1
(43) Pub. Date: Jun. 10, 2010

(54) Total Contact Windowed Slipper with a Lid

(76) Inventor: Rolando Chaparro Cadena, Las Cruces, NM (US)

Correspondence Address: Kenneth Bower, 314 Ironstone Lane, Elverson, PA, 19520, US

(21) Appl. No.: 12/330,814

(22) Filed: Dec. 9, 2008

(51) Int. Cl. A61F 5/00; A61B 19/00

(52) U.S. Cl. 602/27; 602/36; 602/6; 128/898

(57) ABSTRACT

Nearly normal mobility and quality of life, during the treatment of severe surface wounds are supported by the addition of a unique combination of common medical treatment materials with a locally applied body cast. Features of the structure and corresponding kit include off loading and physical protection of the treatment area, evenly applied pressure to maintain a hydrostatically stable environment to promote healing, easy access to the treatment area without removal of most of the structure in addition to the use of readily available inexpensive and space conserving materials for sensitivity to medical cost control and treatment for patients in the developing world, battlefield and other hostile environments.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The invention relates to the area of wound treatment, and more specifically to the area of treatment of ulcers on the sole of the foot.

2. Description of Related Art

Sedentary life style, lack of exercise, excessive consumption of food and drink are among the trends that are causing an epidemic of disease related skin lesions and ulcers. Diabetes leads the list of skin lesion and ulcer causing diseases. Treatment of skin lesions and ulcers requires a locally immobile and protected as well as hydrostatically stable healing environment. The treatments that represent the current state of the art claim to give the patient complete mobility. However in actuality without constant adjustment for swelling and contraction of the extremities the treatments severely hamper a patient's mobility, quality of life and participation in exercise. Therefore, currently known treatments promote the worsening of the underlying disease by promoting increased lethargy, lack of exercise and additional over consumption. This is a degenerating cycle that must be stopped to bring medical costs under control.

The most commonly implemented treatment for a plantar ulcer in the sole of the foot is known as a total contact cast, or TCC. The total contact cast involves completely encasing the foot and lower leg in a cast, providing a bubble area around the foot wound. The first goal of the total contact cast is to protect the foot wound to allow for healing, the second goal of the TCC is to offload the patient's weight from the bottom of the foot by wedging action against the ankle and calf.

The total contact cast method leads to two main problems: First, since the patient's foot wound is isolated, a caregiver must remove the cast to inspect the wound. It is both time consuming and wasteful to remove all of the materials, some of which are rigid. Moreover, the cast must be replaced after inspection, which is also a time consuming process. The second problem is that the total contact cast method greatly decreases a patient's mobility. The cast must be left on for up to eight weeks, leading to a lower quality of life and, potentially, to a further cycle of degrading health.

In addition to the total contact cast, various additional solutions have been proposed in the art to address treatment of plantar ulcers and other foot wounds on the bottom of the foot. In addition, alternatives to the total contact cast have been proposed.

Although recent developments have improved protection of and access to the treatment area of skin lesions and ulcers they neither provide a hydrostatically stable healing environment nor do they support near normal mobility and quality of life for the patient.

A representative sample of state of the art skin lesion and ulcer treatment regimens includes:

The Patent to Sandvig, (U.S. Pat. No. 4,888,225) shows a temporary pliant and extensible splint structure applied to a foot after orthopedic injury. The top of the splint is below the knee and allows for walking with a very elevated walking heel. Sandvig lacks a true contact body cast made of resilient material, has no provisions for accommodating a compress on a wound or ulcer, and the only access to an area on the foot would be by complete removal.

The Patent Application by Rooney (US 2005/0240133) shows a custom made “L” shaped platform having a cavity that relieves pressure from the area of a wound or ulcer on the bottom of the foot. The structure allows for immediate walking after the lengthy process of custom fitting, and incorporates wedges for leveling the sole of the foot with the ground to facilitate an even gate for the patient's comfort.

Rooney lacks use of the much more straight forward and fast application of the contact body cast to immobilize joints adjacent to the wound or ulcer. Moreover, Rooney requires the complete removal of the structure to inspect the healing or change the treatment or compresses. The reliance on adjustable straps to secure the structure to the foot almost certainly allows significant movement and scuffing of the wound or ulcer when taking a step—causing patient discomfort and possible worsening of the conditions being treated.

The Patent to Schuren (U.S. Pat. No. 7,264,604) shows custom foot beds for off loading the treatment area for wounds or ulcers on the bottom of the foot. Schuren provides recesses in the top of the foot bed to off load and provide space for compress materials below the wound or ulcer. Schuren lacks the structure to immobilize the ankle. Moreover, Schuren does not provide separate means to adjust the pressure on the compresses for proper hydrodynamic pressure equalization. Schuren also lacks any provision for walking or leveling of the feet for preventing orthopedic problems due to a crooked gate. There is also a lack of positional stability of the structure relative to the treatment area to provide hydrostatic pressure stability.

The patent to Cavanagh (U.S. Pat. No. 6,610,897) shows an “L” shaped platform with a horizontal surface custom made with a cavity that relieves pressure from the area of a wound or ulcer on the bottom of the foot. The disclosure implies that patients can walk after the custom fitting process. Cavanagh lacks use of the much more stable application of the contact body cast to immobilize joints adjacent to the wound or ulcer as well as requiring the complete removal of the structure to inspect the healing or change the treatment or compresses. The reliance on adjustable straps to secure the structure to the foot almost certainly allows significant movement and scuffing of the wound or ulcer when taking a step. This can cause patient discomfort and possible worsening of the conditions being treated. There is also a lack of positional stability of the structure relative to the treatment area to provide the hydrostatic pressure stability that is key to promoting healing.

The Patent Application to Jensen (US 2008/0039758) shows a combination of the features of the invention to Cavanaugh with the use of the contact body cast with a window for wound or ulcer access. The reliance on adjustable straps to secure the structure to the foot almost certainly allows significant movement and scuffing of the wound or ulcer when taking a step causing patient discomfort and possible worsening of the conditions being treated. Further off loading of the patients body weight during walking by means of ankle struts adds to the likelihood of horizontal motion between the foot bed, wound compresses and the wound causing further patient pain and exacerbation of the condition being treated. There is also a lack of positional stability of the structure relative to the treatment area to provide the hydrostatic pressure stability that is key to the healing process.

### BRIEF SUMMARY OF THE INVENTION

In view of the above-mentioned unfulfilled needs in the prior art, the present invention embodies the objects and advantages detailed herein:

The primary object of the present invention is to provide a structure for the treatment of skin lesions and ulcers that supports near normal mobility and quality of life for the patient while allowing open access to the wound through the structure for treatment and inspection of the lesion.

Another object of the present invention in the case of foot lesions or ulcers is to support near normal patient walking.

A further object of the present invention is to utilize any of a number of contact body cast structures that are known in the art to gain local immobility of the treatment region and additionally provide a window for application of medicines, salves, and compress materials to promote healing.

A further object of the present invention is to reduce by half the time required for a practitioner to apply the structure to a patient.

It is a further object of the present invention to treat patients with multiple conditions including: edema, PAD, stage 3 and 4 ulcers, dermatitis and claustrophobia.

A further object of the present invention is to provide an open system of treatment providing the easiest access to the primary treatment area in addition to access to the surrounding area without destruction of the contact body cast.

A further object of the present invention is to provide for quick disassembly for partial or complete access to the treatment area and area surrounding the treatment area in addition to quick re-assembly after access is completed.

A further object of the present invention is to provide compress materials for application to the skin wound or ulcer to promote healing and for application of medicines, salves or other treatments.

A further object of the present invention is to provide a protective layer that applies an evenly distributed force across the treatment area providing hydrostatic pressure stability that is critical to the promotion of accelerated healing.

A further object of the present invention is to fasten the protective layer to the contact body cast structure with fastening member(s) that make the protective layer easy to remove for monitoring the progress of healing or for the application of additional treatments.

A further objective of the present invention is to make the protective layer load bearing where necessary such as with wounds or ulcers on the bottom of the foot.

A further object of the present invention is to provide a surface to the outside of the protective layer that is suitable to provide the cushioning, traction and load bearing functions so that the patient can walk.

A further object of the present invention is to be able to easily adjust the thickness of the protective layer cushioning and traction surface and or the sole of the shoe worn on the patient's healthy foot to equalize the stance of the individual thus avoiding secondary injuries to the patient's body.

A further object of the present invention is for the protective layer cushioning and traction surface to protect the body cast, protective layer and fastening members from the wear and tear of contact with floors and sidewalks.

A further object of the present invention is to make the protective layer traction element from any of a variety of resilient materials, shapes and configurations taking into consideration at least one of physical properties, availability, stability in the local environment, environmental consciousness and cost.

A further object of the present invention is make the protective layer traction element of various thicknesses by adding or subtracting layers or adjustment of the thickness of a single layer.

A further object of the present invention is to make the protective layer from any of a variety of semi-rigid materials, shapes and configurations taking into consideration at least one of physical properties, availability, stability in the local environment, environmental consciousness and cost.

A further object of the present invention is to make the fastening members at least partially of any of a variety of elastic or resilient materials in a number of potential shapes and configurations that will provide a relatively evenly applied force between the body contact cast and the protective layer taking into consideration at least one of physical properties, availability, stability in the local environment, environmental consciousness and cost.

A further object of the present invention is to provide a kit containing the materials to apply a contact body cast with a window, compress materials, protective layer, fastening member(s), traction elements and instructions for application and use.

A further object of the present invention is to make the kit small for ease of distribution to the third world, war theater and other hostile environments.

A further object of the present invention is to promote physical activity and exercise on the part of diabetes patients suffering from skin wounds and ulcers to break the degenerating cycle brought on by the use of current state of the art treatment structures and treatment regimens.

A further object of the present invention, disclosed as the preferred embodiment is to promote walking on the part of diabetes patients suffering from skin wounds and ulcers to break the degenerating cycle brought on by the use of current state of the art treatment structures and treatment regimens.

The preferred embodiment of the present invention is a foot wound dressing system that is comfortable for the patient to wear. In practice, a caregiver could retrieve all of the necessary materials from a kit which is provided with application instructions. In the preferred embodiment, the kit would contain: cotton gauze, a plaster or fiberglass foot cast materials, a gauze roll bandage, various wound contact dressing materials, a felt covered fiberglass splint, rubber sole material, adhesive glue, and an elastic bandage.

In operation, the caregiver would first use the cotton gauze to form a bottom layer, which would be wrapped around the foot, the ankle, and the lower leg. The bottom layer would provide full coverage with exception of the wound. Next, a rigid layer is added using the plaster or fiberglass foot cast materials. The rigid layer would cover from the ankle toward and covering the foot, with the toes left exposed. As with the bottom layer, the rigid layer would not cover the wound. Instead, a window would be left in the area of the wound. Various methods could be used to provide the window. For example, the caregiver could simply form the window by hand before the casting material hardens. Or the caregiver could cut the window into the rigid layer after hardening. Once the rigid layer is in place with the window open over the wound, a gauze roll bandage is wrapped around the rigid layer, the ankle and the lower leg to secure the rigid layer in place. Again, the gauze roll bandage does not cover the rigid layer window. Next, the wound is dressed with the caregivers dressing of choice. This could include various cotton gauze layers, bandages or ointments. Next, a felt covered fiberglass splint is added from the heel to the toes. The felt covered fiberglass splint provides additional rigidity to the system as the patient ambulates. In addition, the felt covered fiberglass splint holds the wound dressing in place, and is removable. Next, the caregiver can add rubber sole material to various portions of the felt covered fiberglass splint. The rubber sole material provides traction when the patient walks on a ground surface. The caregiver can select from various thicknesses of rubber sole material so that leg length equalization can be achieved. Leg length equalization is important in avoiding additional disease conditions such as charcot joint. Finally, the felt covered fiberglass splint is secured in place by wrapping an elastic bandage around the splint. The caregiver would not wrap the elastic bandage in the areas of the rubber sole material.

The patient may now comfortably walk around. And when the time comes for the wound to be re-dressed, the caregiver need only disassemble the elastic bandage and the felt covered splint. The caregiver may then remove the wound dressings and inspect the wound. If additional healing time is required, the wound may be re-dressed, and the felt covered splint can be re-assembled along with the elastic bandage. This type of assembly and disassembly could be performed multiple times each day, as necessary, by the simple removal of the elastic bandage and the fiberglass splint.

### BRIEF DESCRIPTION OF THE SEVERAL VIEWS OF THE DRAWING(S)

Various other objects, features and attendant advantages of the present invention will become fully appreciated through consideration of the accompanying drawings and the detailed description following, in which like reference characters designate the same or similar parts throughout the several views, and wherein:

FIG. 1(a) is a prior art frontal view of a treatment for foot wounds or ulcers,

FIG. 1 (b) is a frontal view of a treatment of the present invention for foot wounds or ulcers.

FIG. 2(a) is a top view of a prior art foot wound or ulcer treatment kit.

FIG. 2(b) is a top view of the kit of the present invention.

FIG. 3(a) is a view from below the side of a foot with a skin ulcer.

FIG. 3(b) is a view from below the side of a foot in a first layer of a contact body cast with a window.

FIG. 3(c) is a view from below the side of a foot in a second layer of a contact body cast with a window.

FIG. 3(d) is a view from below the side of a foot in a third layer of a contact body cast with a window.

FIG. 3(e) is a view from below the side of a foot in a contact body cast with a compress applied to the skin ulcer.

FIG. 3(f) is a view from below the side of a foot in a contact body cast with a protective cover applied over the compress.

FIG. 3(g) is a view from below the side of a foot in a contact body cast with a traction member applied to the protective cover.

FIG. 3(h) is a view from below the side of a foot in a contact body cast with a fastening member applied over the protective cover.

FIG. 3(i) is a view from below the side of a foot in a contact body cast with the traction member being adjusted for height.

### DETAILED DESCRIPTION OF THE INVENTION

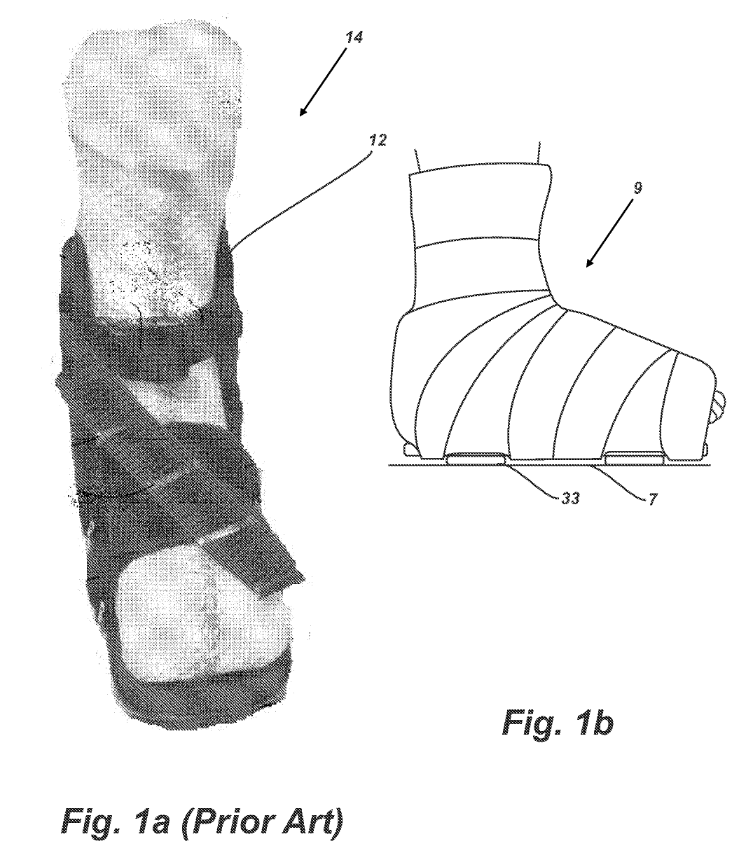

FIG. 1(a) shows a patient receiving a prior art treatment 14 for a wound or ulcer on the bottom of the foot. The patient's foot and lower leg is covered to the knee in a contact body cast. The contact body cast is in turn covered by an elaborate boot 12. The patient's mobility and quality of life are limited by the use of crutches. The patient's body structure is contorted by the un-natural and un-even weight distribution between the good foot and the afflicted foot.

FIG. 1 (b) shows a patient receiving the treatment of the present invention for a wound or ulcer on the bottom of the foot. The patient's foot up to the ankle is covered in a two piece contact body cast. The cast is in turn covered by a protective cover that in turn provides one or more traction members 33 for contact with walking surfaces 7. The patient's mobility and quality of life are sustained by natural walking largely due to allowing of movement of the patient's ankle. A cane or walking stick may be used to aid with balance. The patient's body structure is un-affected due to the natural position of walking, the light weight of the treatment structure 9 and the maintenance of natural structural position of the hips and spine by proper adjustment of the thickness of traction member 33.

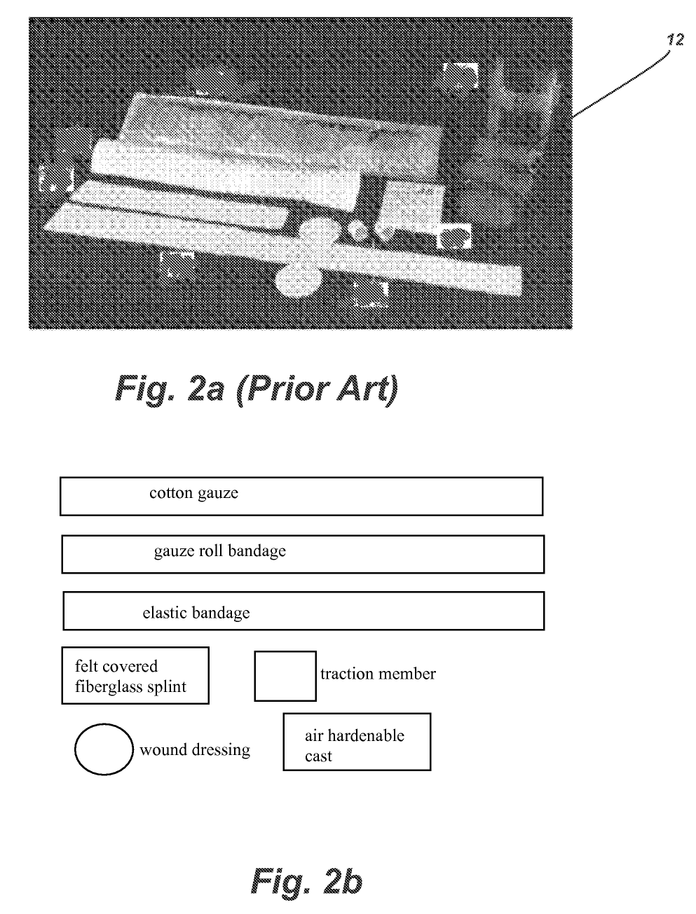

FIG. 2(a) shows the prior art kit of the recent US patent Application to Jensen (US 2008/0039758). The elements of note are the boot 12, which should be stocked in various sizes to assure proper fit with various size feet, which adds to the cost of the kit. Lastly, the size of the box for the kit is sizable contributing to high transportation and storage costs and vulnerability to damage. Excessive packaging also raises issues of disposal and its environmental effects.

FIG. 2(b) shows the present invention in kit form. The elements of note are the felt covered fiberglass splint and elastic bandage. Despite their very compact size and obviously lower cost, the felt covered fiberglass splint and elastic bandage provide the superior function of hydrostatic pressure stability on the wound or ulcer as well as allowing the patient to walk.

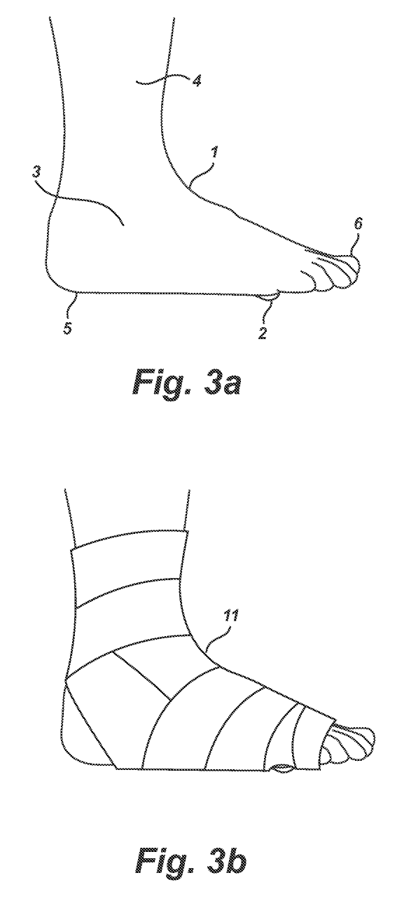

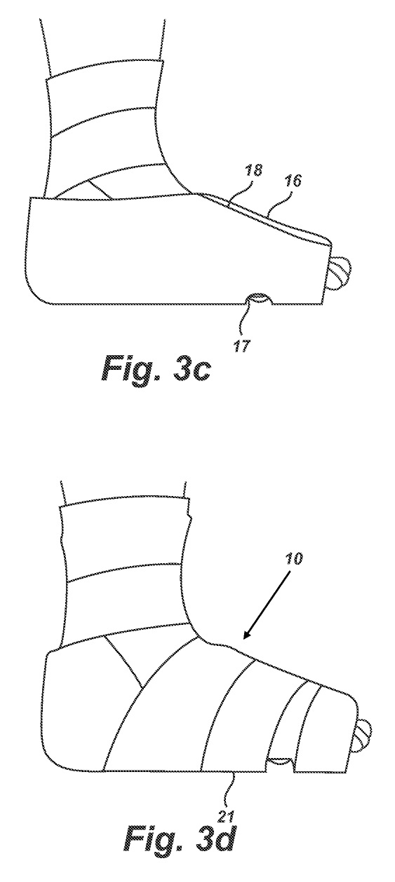

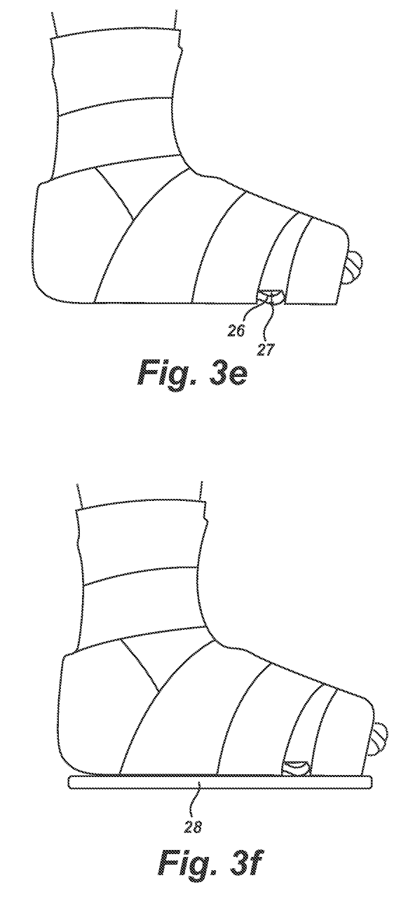

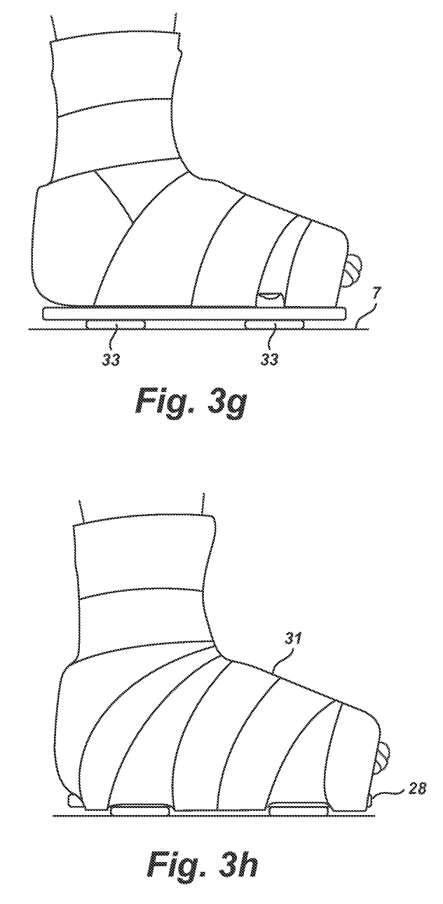

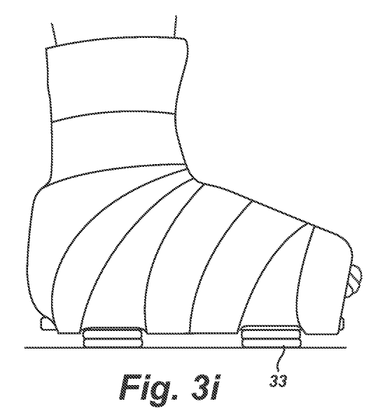

FIG. 3(a) is a view from below the side of a foot 1 with a skin ulcer 2, or area. The bottom of the foot 1 is a common location for a diabetes related ulcer 2 because of both neurologic and vascular complications in this area.

Referring now to FIG. 3(b-d), multiple views are shown from below the side of a foot 1 in a contact body cast 10 with a window 17. As shown in FIG. 3b, the inventor prefers to use cotton gauze for application of a cushioning layer 11, or protective material, around the foot 1 and leg 4. Next, as seen in FIG. 3c, this is followed by application of air curable sheet of body cast material 16 to form the contact body cast 10, followed by cutting of the window 17 around the afflicted area (un-shown). A preferred method of applying the body cast forms two halves, one on either side of the foot. The two halves are easily removed and replaced in position on the foot without destroying the cast before and after full access to the area surrounding the treatment area. A parting line 18 is shown in FIG. 3c, although this parting line 18 could be provided in various locations, depending upon the location of the wound and any other factor considered by the practitioner. As seen in FIG. 3d, the contact body cast is secured by a gauze roll bandage 21. The contact body cast 10 could be formed using numerous alternative methods that are considered to be within the spirit of the instant invention.

FIG. 3(e) is a view from below the side of a foot 1 in a contact body cast 10 with a compress 26 applied to the skin ulcer. Like the contact body cast, there are a number of alternative compress materials, configurations and medicines or salves available for this purpose all of which are considered to fall within the spirit of the present invention. However, the configuration that provides optimum affect of the hydrostatic pressure stability on the ulcer is for the compress 26 to have a thickness 27 approximately matching the depth of the window 17 and a shape and size approximating the shape and size of the window 17. One of the major problems that is addressed by the present invention is treatment of patients with multiple problems such as edema, PAD, stage 3 and 4 ulcers, dermatitis and claustrophobia which require access to the entire area surrounding the treatment area. The two part contact body cast of the preferred embodiment facilitates this access without destroying the cast and it permits fast and easy replacement of the entire treatment structure.

FIG. 3(f) is a view from below the side of a foot 1 in a contact body cast 10 with a protective member 28 applied over the compress 26. The protective member 28 material shown is semi-rigid and made of fiber-glass. A well known source of fiberglass sheets in the medical field is a splint material that is commonly available with one or more sides covered in felt. The preferred configuration of the protective member 28 is shown and covers the entire bottom of the foot 1. However, it is not mandatory that there be any particular relationship between the size and shape of the protective covering or its relationship to the shape or size of the contact body cast 10 or foot 1 or any other body part being treated (un-shown).

FIG. 3(g) is a view from below the side of a foot 1 in a contact body cast 10 with a traction element 33 applied to the protective member 28. The application of the traction member(s) 33 to the protective member 28 can take place at any point in the assembly of the treatment structure 9. This being the case, it is preferred that the traction elements 33 be placed to avoid excessive deflection of the protective cover during patient walking. Experience shows that use of traction members placed below the heel bone and the metatarsal bones of the foot 1 provides good results. However any other number, size or placement of traction element(s) is considered to be within the spirit of the present invention.

FIG. 3(h) is a view from below the side of a foot 1 in a contact body cast 10 with an elastic band 31 applied over the protective member 28. In the preferred embodiment of the present invention the elastic band 31 is wrapped while being placed in nearly constant tension around the top and sides of the contact body cast 10. The winding of the elastic band 31 can be routed between the traction elements 33 giving freedom to traction members 33 placement. Moreover, the elastic band 31 holds the protective member 28 in place and resists the downward forces of the hydrostatic pressure of the treatment area on the top of the protective member 28. One of ordinary skill could devise other fastening members either wholly or in part made of resilient or elastic materials that are considered to be within the spirit of the present invention.

FIG. 3(i) is a view from below the side of a foot 1 in a contact body cast 10 with the traction members being adjusted for height. Addition of additional layers of traction elements 33 is the simplest method of adjusting the total height of the treatment structure. Application of traction element(s) to the footwear of the patient, should the total height of the treatment structure raise the patients body structure in an un-natural position, is also very convenient (un-shown). However, supply of protective covers with layers made up of a plurality of traction members that can be peeled off for height adjustment or any means of adjusting the height of a single traction element are considered to be within the spirit of the present invention.

It is to be understood that the invention is not limited in its application to the details of construction and to the arrangements of the components set forth in the preceding description or illustrated in the drawings. The invention is capable of other embodiments and of being practiced and carried out in various ways. Also, it is to be understood that the phraseology and terminology employed herein are for the purpose of the description and should not be regarded as limiting.

1. A structure for treatment of an area of a patient, comprising:
a contact body cast;
a window in the contact body cast for exposing the area;
a compress in the window for covering the area;
a protective member for covering the compress and covering all of part of the contact body cast;
a fastening member for holding the protective member in contact with the compress and the contact body cast.

2. The structure of claim 1, wherein:
the contact body cast is made of a plurality of sections for ease of removal and subsequent re-positioning over the area.

3. The structure of claim 1, wherein:
wherein the contact body cast allows normal functioning of the ankle.

4. The structure of claim 1, wherein:
the area is on a bottom of a foot.

5. The structure of claim 1, wherein:
the patient can walk without external support.

6. The structure of claim 1, wherein:
the protective member is made of a semi-rigid material.

7. The structure of claim 4, wherein:
the semi-rigid material is fiber-glass.

8. The structure of claim 1, wherein:
the protective member is made from a felt covered fiber-glass splint.

9. The structure of claim 1, wherein:
the fastening member is at least in part made of resilient or elastic material.

10. The structure of claim 7, wherein:
the fastening member is made of an elastic bandage material.

11. The structure of claim 1, further comprising:
one or more traction elements attached to the protective member.

12. The structure of claim 1, wherein:
the protective member and traction elements are integral.

13. The structure of claim 1, wherein:
the fastening member and protective cover are integral.

14. The structure of claim 1, wherein:
the protective member, traction elements and fastening elements are integral.

15. The structure of claim 9, wherein:
the thickness of the traction elements is used to adjust the height of one or both of the patient's legs.

16. A method for providing a covering for an area under treatment on a dermis comprising the following steps in any order:
applying a contact body cast to the dermis surrounding the area under treatment;
forming a window in the contact body cast for access to the area under treatment;
applying a compress over the area under treatment;

17. The method of claim 16, wherein the contact body cast is applied in a plurality of sections to provide open access to the treatment area and the area surrounding the treatment area.

18. The method of claim 16, wherein the contact body cast allows normal functioning of the ankle.

19. The method of claim 16, further comprising the following step in any order:
applying a semi-rigid member covering at least the area of the window.

20. The method of claim 16, further comprising the following step in any order:
applying a fiberglass splint material covered by felt over at least the area of the window.

21. The method of claim 16, further comprising the following step in any order:
applying a resilient material to the protective cover to provide traction with a walking surface.

22. The method of claim 16, further comprising the following step in any order:
applying an elastic band to hold the protective cover and traction element to the contact body cast,

23. The method of claim 16, further comprising the following step in any order:
apply one or more additional traction element layers to either the treatment structure or to the foot-ware on the patients good foot to improve the patients gait.

24. A kit for treating a wound or ulcer on the bottom of a foot for allowing the patient to walk without causing further injury comprising:
a protective material for separating the dermis from body cast material;
a body cast material;
a semi-rigid protective cover material;
an elastic or resilient fastening material;
one or more resilient traction elements;
a set of instructions for application of the materials for treatment of the dermis.
